(19) United States
(12) Patent Application Publication
(10) Pub. No.: US 2005/0013821 A1
(43) Pub. Date: Jan. 20, 2005

(54) Mint extract and related pharmaceutical compositions

(76) Inventor: Susan Anton, Stanmore (GB)

Correspondence Address: WILLIAM COLLARD;COLLARD & ROE, P.C., 1077 NORTHERN BOULEVARD, ROSLYN, NY, 11576, US

(21) Appl. No.: 10/496,437

(22) PCT Filed: Nov. 20, 2002
(86) PCT No.: PCT/GB02/05225

(30) Foreign Application Priority Data
Nov. 20, 2001 (GB) 0127720.1

(51) Int. Cl. A61K 39/00; A61K 39/38

(52) U.S. Cl. 424/184.1

(57) ABSTRACT

A pharmaceutical composition comprises extract (or synthetic or other equivalent thereof) of one or more members of the mint (mentha) family, e.g. peppermint, pennyroyal, spearmint (mint), e.g. menthol or peppermint oil, for use as an active substance for topical application to the external skin area in the vicinity of the vulva or anus, and is applied for alleviating burning pain sensations in such vicinity, or alleviating pain sensations in the urethra or rectum, such pain sensations being of a kind usually occurring upon passing acid urine or faeces and usually associated with what is popularly called cystitis or irritable bowel syndrome; or again comprises an emollient, e.g. comprising liquid paraffin and/or white soft paraffin, and extract (or synthetic or other equivalent thereof) of one or more members of the mint (mentha) family, for topical application to the external skin area, and is applied for alleviating severe pains, e.g. due to shingles or generally where there is severe pain, e.g. skin pain, possibly for burns. The composition can be embodied in a gel, though preferably in a cream, ointment, wipe or spray. The compositions can be applied, with novel effect to a further condition, comprising cuts, abrasions or other wounds of the skin. Topical application has been found to accelerate the healing thereof.

### FIELD OF THE INVENTION

This invention relates to pharmaceutical compositions.

### BACKGROUND

A frequently occurring condition that women suffer during and after urination is a burning pain at the external skin area in the vicinity of the vulva (which term is used herein to denote the external skin area in women in the region of the external opening of the urethra). The word “at” is used to denote that the pain may be felt below the skin surface. (References herein to adults comprehend references to children. References herein to human beings comprehend references to animals where the context allows.)

This condition is popularly called cystitis and is a condition that is suffered by many millions of women of all age groups all over the world. One authority estimates that one in four women will suffer from cystitis at some point in their lives. Cystitis is inflammation of the bladder, usually due to infection, but the term is popularly used to cover a range of infections and irritations in the lower urinary system. These are usually associated with a burning sensation during urination and a frequent need to urinate, and this can occur at any time without any warning.

Most such infections are due to one type of bacteria—E. coli—which normally lives in the bowel and causes no trouble, but which can sometimes reach, and cling to and infect, the external (vulval) opening of the urethra, the urinary system.

There are a number of products available to treat cystitis. Alkalising the urine helps to eradicate the germs and also soothe the bladder, and diuretics help cleanse the system. Antibiotics are used to kill the bacteria.

The many treatments that are available to be administered orally take time (often several hours) to have any effect in alleviating the burning pain and do not help immediately, and the various creams on sale for use in the relevant skin area do not appear to have any substantial effect.

### THE INVENTION

The present inventor found that a particular substance (peppermint oil, oleum menthae piperitae) could be externally applied to give instant relief from these burning pains, and this was associated with giving a continuing substantial and long-term relief. Further research showed that related substances (extracts from members of the mint family) had similar effect, some less and one (menthol) substantially more. These substances have not previously been applied for this purpose and in this manner. The efficacy of these substances suggested to the inventor that they might be useful in another, somewhat similar, situation, i.e. the burning pains often occurring in the anal region of men and women in association with irritable bowel syndrome (IBS), particularly upon passing faeces, and this was borne out by research.

While menthol is known for application to other parts of the body, more especially for respiratory troubles, it is not known in a suitable composition for use by topical application in the immediate vicinity of the vulva or anus, nor for use in treating pain sensations in such vicinity.

There was an indication that these substances, particularly when embodied in an emollient composition, e.g. comprising liquid paraffin and/or white soft paraffin, would have a beneficial effect in relation to shingles, whether in the vulval or anal areas or elsewhere. In this case, the action is thought to comprise a combination of the anaesthetic and the soothing and/or healing properties of the substance and (in the emollient composition) a special action due to the substance being dissolved in the emollient component/s, e.g. the paraffin/s. Part of this special action appears to be that the emollient paraffin inhibits, or controls, the release of the substance's powerful vapours, and/or that it has a synergistic effect with the substance and/or its vapours. This suggests that such emollient composition (whether as cream, ointment, wipe or spray, see below) may also be beneficial for, and when applied to, the external skin area of other parts of the body where there is severe pain, especially for example skin pain, possibly even for burns.

According to one aspect of the invention, there is provided a pharmaceutical composition, characterised in that it comprises extract (or synthetic or other equivalent thereof) of one or more members of the mint (mentha) family for use as an active substance for topical application to the external skin area in the vicinity of the vulva or anus.

In these days of rapid research into the synthesis of naturally occurring chemicals, and of equivalent chemicals having related or somewhat similar properties, the substance would appear of equal relevance whether it is actually an extract of the natural plant or whether it is a synthetic or other equivalent thereof.

According to another aspect of the invention, there is provided a pharmaceutical composition, characterised in that it comprises an emollient and extract (or synthetic or other equivalent thereof) of one or more members of the mint (mentha) family for topical application to the external skin area.

According to another aspect of the invention, there is provided a pharmaceutical composition, characterised in that it comprises, as a pharmaceutically active substance, extract (or synthetic or other equivalent thereof) of one or more members of the mint (mentha) family in a concentration and form, and in a formula of the composition, suitable for topical application to the external skin area in the vicinity of the vulva or anus.

According to another aspect of the invention, there is provided a pharmaceutical composition, applied topically in the vicinity of the vulva or anus, alleviating burning pain sensations in such vicinity, or alleviating pain sensations in the urethra or rectum, such pain sensations being of a kind usually occurring upon passing acid urine or faeces and usually associated with what is popularly called cystitis or irritable bowel syndrome, which composition comprises extract (or synthetic or other equivalent thereof) of one or more members of the mint (mentha) family.

According to another aspect of the invention, there is provided a pharmaceutical composition, characterised in that it comprises extract (or synthetic or other equivalent thereof) of one or more members of the mint (mentha) family for use as an active substance for topical application to the external skin area in the vicinity of the vulva or anus for the purpose of alleviating burning pain sensations in such vicinity, or for the purpose of alleviating pain sensations in the urethra or rectum, such pain sensations being of a kind usually occurring upon passing acid urine or faeces and usually associated with what is popularly called cystitis or irritable bowel syndrome.

According to another aspect of the invention, there is provided a pharmaceutical composition, characterised in that it comprises, as a pharmaceutically active substance, extract (or synthetic or other equivalent thereof) of one or more members of the mint (mentha) family in a concentration and form, and in a formula of the composition, suitable and effective for topical application to the external skin area in the vicinity of the vulva or anus for the purpose of alleviating burning pain sensations in such vicinity, or for the purpose of alleviating pain sensations in the urethra or rectum, such pain sensations being of a kind usually occurring upon passing acid urine or faeces and usually associated with what is popularly called cystitis or irritable bowel syndrome.

The efficacy of the compositions is not necessarily only for those pain sensations actually caused by passing acid urine or faeces, or cystitis or IBS, but is also for alleviating pain sensations of similar kind in such vicinity.

Members of the mint family which can be used: e.g. peppermint, pennyroyal, spearmint (mint). Suitable extracts: e.g. menthol, peppermint oil.

In the preferred composition, the extract or equivalent comprises menthol or its equivalent and/or peppermint oil or its equivalent.

The composition may comprise the extract or equivalent up to 10% w/w but it is preferably present in a concentration of not more than substantially 5% w/w of the composition. About 5% w/w gives good results, but for some individuals a lower concentration is appropriate, usually in the range of 2% to 5% w/w of the composition, but in some cases as low as substantially 0.5% w/w of the composition, and even substantially 0.0005% w/w of the composition, has been found to give some relief.

The composition can be embodied in a gel, cream or ointment, or a wipe, or a spray. Such forms of embodiment can give immediate relief on application to the burning pain, can be easily and readily available at any time, and can be applied at any time and be easily carried.

According to another aspect of the invention, there is provided the use as an active therapeutic substance of extract (or the synthetic or other equivalent thereof) of one or more members of the mint family for the manufacture of a pharmaceutical composition for topical application to the external skin area in the vicinity of the vulva or anus for alleviating burning pain sensations in such vicinity of a kind usually occurring upon passing acid urine or faeces and usually associated with what is popularly called cystitis or irritable bowel syndrome.

In such use, said extract or equivalent may comprise menthol or its equivalent and/or peppermint oil or its equivalent, preferably in a concentration as indicated above, and preferably embodied in a form as indicated above.

According to another aspect of the invention, there is provided the use of a pharmaceutical composition for the treatment of the body by therapy by topical application in the vicinity of the vulva or anus, characterised in that the composition is any of the compositions indicated above according to the invention.

In the sense that the relief of pain by the composition in some circumstances is purely a temporary palliative to alleviate pain and not associated with a continuing substantial and long-term relief by effecting in substance a cure or healing, the use of the composition may be considered to be non-therapeutic. According to another aspect of the invention, there is provided the use of a pharmaceutical composition for the non-therapeutic treatment of the body by topical application in the vicinity of the vulva or anus, in which the composition is any of the compositions indicated above according to the invention.

According to another aspect of the invention, there is provided a method of treating the human body, which comprises applying to an external skin area a pharmaceutical composition comprising an emollient and said extract or equivalent.

According to another aspect of the invention, there is provided a method of treating the human body, which comprises applying to an external skin area in the vicinity of the vulva or anus a pharmaceutical composition as indicated above according to the invention.

Trials have been carried out according to, or using, each of the aforementioned aspects of the invention.

### MORE PARTICULAR DESCRIPTION

Reference will now be made, by way of further example, to trials that have been carried out.

Successful initial trials were carried out with peppermint oil in a gel containing, inter alia, stearate and essential oil ingredients. Peppermint oil was also tried, successfully, in a cream containing liquid paraffin and white soft paraffin.

Much improved relief was obtained using menthol instead of peppermint oil. This was suitably soluble (at least 10% w/w) in liquid paraffin, white soft paraffin and essential oils as ingredients of a suitable composition embodied in a cream or ointment.

Trials were carried out with female and male subjects. Two series of trials, each on one patient, are given by way of example. In these, the same composition of a cream embodying the invention was used. This comprised 1.5 g of menthol triturated in a mortar into 30 g of base cream. The menthol used was an extract of a natural product, and in the form of long (about 1.5 cm) crystals, obtained as a standard pharmaceutical product, BP or EP. The base cream was a standard product for use as a base cream in “sensitive skin areas” obtainable from Schering-Plough, e.g. from Schering-Plough Limited of Welwyn Garden City, Hertfordshire AL7 1TW, UK, under their name “Dipro-base” (Registered Trade Mark) cream. Its formula is given by them as: chlorocresol, cetomacrogol, cetostearyl alcohol, liquid paraffin, white soft paraffin, phosphoric acid, sodium dihydrogen phosphate, purified water, and it is stated to be a soothing, emollient and moisturising cream, (though it had no noticeable effect on the burning pains when tried without the menthol.)

Trials, Series A

The subject was a female who had cystitis and suffered from burning pains at the external skin area in the region of the vulva, more particularly upon passing water (urine). In six trials, upon occurrence of the burning pains, a sample of the cream was applied in the area with a finger. The result was the same in every trial. There was instant complete relief from the burning. pains, within an estimated 1 to 2 seconds, certainly less than 5 seconds. This was coupled with substantial relief of associated pain extending back along the urethra, sometimes back to the bladder. The mechanism of operation is believed to be: anaesthetic action against the burning pains at the external skin followed quickly by penetration of the urethra by the powerful aromatic vapours of the menthol causing further anaesthetic action against the pain within the urethra and possibly the bladder, both actions possibly being coupled with a soothing action of the menthol and its vapours. As described by the subject, the “powerful and stimulating glow” and relief from pain could be felt progressing back up the urethra. These effects lasted for approximately 15 minutes, after which there was usually continuing and long-term relief, the burning pains being completely cured or very much relieved.

Trials, Series B

The subject was a male who had Irritable Bowel Syndrome and suffered from burning pains at the external skin area in the region of the anus, particularly upon passing a motion (faeces). In two trials, upon occurrence of the burning pains, a sample of the cream was applied in the area with a finger. The result was the same in both trials. There was instant complete relief from the burning pains, within an estimated 1 to 2 seconds, certainly less than five seconds. This was coupled with substantial relief of associated pain extending back along the rectum to the bowel. The mechanism of operation is believed to be similar to that mentioned above in relation to Series A of Trials, reading rectum and bowel for urethra and bladder. Likewise, the “stimulating glow” and relief from pain could be felt progressing back up the rectum. Again, the relief lasted for approximately 15 minutes, after which there was usually continuing and long-term relief, the burning pains being completely cured or very much relieved.

(In other trials than the Series A and B mentioned above, with a composition having the active ingredient less concentrated, or using a weaker member of the mint family, there was no continuing and long-term relief obviously caused by the composition after 15 minutes, by which time the composition had apparently ceased to act, but the pain had nevertheless somewhat naturally abated, apparently due to the body's natural reactions. This may be considered a non-therapeutic action.)

Additional to these trials, compositions were prepared using menthol (and others using peppermint oil) embodied in a light cream (which included water) with which small pieces of soft cloth were impregnated to form wipes. These were just as successful, sometimes being preferred because the finger did not have to touch the external skin directly. Other menthol or peppermint oil compositions were embodied in a spray which included glycols, glycerol and ethanol, and were used successfully. Alternative combinations of ingredients could be utilised, e.g. as known to those skilled in the art, possibly including a pharmaceutically acceptable excipient for the active ingredient/s. Preferably, the composition should contain one or more of vitamins E, A and D combined with a moisturizing element in white soft paraffin form (obtained as “Vaseline”, Registered Trade Mark in the UK) or aloe form for delicate skins. The composition should preferably be formulated so that it is effectively greaseless and non-staining. It should not contain hexachlorophene.

It is to be noted that there may be occasions when it is socially or physically impossible or very difficult to apply a quick cure or palliative (like washing). Often, even on a bidet, there is only temporary relief while irrigating the area, and this relief stops when ceasing to irrigate the area. On the contrary, the present examples of compositions are very effective in such a case to produce instant relief (i.e. in less than 5 seconds), and then to give continuing, long-term relief.

Further aspects and embodiments of the invention will be as recited in the accompanying claims.

Further researches have shown that the compositions mentioned herein can be applied to a further condition, with novel effects. Said condition comprises cuts, abrasions or other wounds of the skin. Topical application has been found to accelerate the healing of the skin, such that a minor cut can thereby be completely healed within one day (12 hours) or even half a day, or less. The composition is introduced, e.g. rubbed well and/or deep, into the wound, and appears to pull its sides together while accelerating the healing of the wound by regeneration of the bonding between the sides. This was done to a wound, a cut on the front of a finger, 10 mm long and reaching 3 mm deep; the sides healed firmly together in 12 hours and there was no sign of scar in 24 hours. At the same time, pain of the wound is reduced. Thus, another aspect of the invention provides the use as an active therapeutic substance of extract (or the synthetic or other equivalent thereof) of one or more members of the mint family in the manufacture of a medicament for the therapeutic treatment of skin wound; and a further aspect of the invention provides the use as an active therapeutic substance of extract (or the synthetic or other equivalent thereof) of one or more members of the mint family in the manufacture of a topical medicament for the therapeutic treatment of skin wound to ameliorate the healing thereof.

Where the phrase “mint family” is used hereinabove, further researches have shown that this could be extended to terpenes.

It will be apparent to one skilled in the art, that features of the different embodiments disclosed herein may be omitted, selected, combined or exchanged and the invention is considered to extend to any new and inventive combination thus formed.

1-46. (Canceled)

47. A pharmaceutical composition for use in treating cystitis or irritable bowel syndrome comprising:
an extract or synthetic form of one or more members of a mint or Mentha family wherein said composition is for use as an active substance for topical application in the vicinity of a user's vulva or anus.

48. A method for treating cystitis comprising the steps of:
applying a pharmaceutical composition comprising an extract or synthetic form of one or more members of a mint or Mentha family wherein said application is to or in a vicinity of a user's vulva or anus.

49. The method as in claim 48, wherein said step of applying said pharmaceutical composition includes applying said composition topically to a user's skin.

50. The composition as in claim 47, wherein said extract or equivalent comprises menthol or its equivalent.

51. The composition as in claim 47, wherein said extract comprises peppermint oil or its equivalent.

52. The composition as in claim 47, wherein said extract or equivalent includes a concentration of not more than 5% w/w of the entire composition.

53. The composition as in claim 52, wherein said extract or equivalent is present in a concentration range of from 2% to 5% w/w of said composition.

54. The composition as in claim 47, wherein said composition is in the form of a cream.

55. The composition as in claim 47, wherein said composition is in the form of a wipe.

56. The composition as in claim 47, wherein said composition is in the form of a spray.

57. The method as in claim 49, wherein said step of applying a pharmaceutical composition includes spraying said composition on a user's skin.

58. The method as in claim 49, wherein said step of applying a pharmaceutical composition includes wiping said composition on a user's skin.

59. The method as in claim 49, wherein said step of applying a pharmaceutical composition includes applying a cream of said pharmaceutical composition to a user's skin.

60. The method as in claim 49, wherein said extract comprises peppermint oil.

61. The method as in claim 49, wherein said extract comprises menthol.

62. The method as in claim 49, wherein said extract or equivalent includes a concentration of not more than 5 wt % of said composition.

63. The method as in claim 49, wherein said extract or equivalent is present in a concentration range of from 2 to 5 wt % of said composition.
